(19) United States
(12) Patent Application Publication
(10) Pub. No.: US 2006/0216365 A1
(43) Pub. Date: Sep. 28, 2006

(54) Aromatherapeutic environmental system

(76) Inventors: Monica R. Nassif, Minneapolis, MN (US); Pam Helms, Minneapolis, MN (US)

Correspondence Address: Mark A. Litman & Associates, P.A.;York Business Center, Suite 205, 3209 West 76th St., Edina, MN, 55435, US

(21) Appl. No.: 11/441,647

(22) Filed: May 26, 2006

Related U.S. Application Data
(63) Continuation of application No. 09/659,502, filed on Sep. 11, 2000.

(51) Int. Cl. A61K 36/752; A61K 36/61; A61K 36/53; A61K 36/537; A61K 36/534; A61K 36/899; A61K 36/896; A61K 36/48; A61K 36/28; A61K 36/23; A61K 36/32

(52) U.S. Cl. 424/736; 424/745; 424/746; 424/747; 424/750; 424/769; 424/764; 424/757; 424/753; 424/776; 424/778; 424/765; 424/742

(57) ABSTRACT

A household function and aromatherapy is provided in a single product, which may also provide non-synthetic antibacterial activity by a method for providing aromatherapy to persons within an ambient environment comprising applying a liquid composition to an inanimate surface to effect a household function, the liquid composition comprising an aromatherapeutic concentration of an aromatherapeutic essential oil, completing the household function, allowing the aromatherapeutic essential oil to remain within the ambient environment to effect aromatherapy on persons or animals within the ambient environment. A kit for providing both a household function and aromatherapy comprises different household products for effecting aromatherapy to an ambient environment, the kit containing at least three liquid household products, each household product comprising a liquid composition comprising at least 0.2% by total weight of an antibacterial essential oil and a separate ingredient that effects a household function selected from the group consisting of surface cleaning, starching, polishing, or finishing, the essential oil in each household product within the kit comprising at least one identical essential oil.

### BACKGROUND OF THE INVENTION

1. Field of the Invention

The present invention relates to the field of household systems and compositions for general application around the home or workplace and their use to provide aromatherapeutic and environmentally friendly materials for use around the home or workplace. The compositions also may comprise antimicrobial activity from the underlying components.

2. Background of the Art

The concept of the use of sensory effects to modify behavior or emotional condition is probably better accepted than is commonly known, or would be if general knowledge were considered. Everyone appreciates that depravation of audio stimulation (sound) is conducive to an ability to relax, as is often necessary before sleeping. Appropriately directed visual stimulation can also be used to initiate or enhance targeted emotions. Motion picture directors and cameramen specifically design images to assist in conveying specific emotions to a viewer, with all emotions from terror to passion to sorrow within the artistic control of a skilled artisan.

Pavlov's studies of audio stimulation and response Supports the concept that a specific physiologic stimulation can produce targeted results in animals, including stimulating emotional states of the animal.

Even the well known words of William Shakespeare from Romeo and Juliet, should be read with a strong inkling of aromatherapy. The words in Act I, Scene IV: “That which we call a rose, by any other name would smell as sweet.” mean more than the stimulus of the rose upon a nose merely being recognition of an effect associated with olfactory recognition of a generic class of carbohydrates (sugars), with the property of “sweet” being the specific objective and resulting quality of the stimulus. The words should actually indicate that, without regard to any other physiological stimulus (e.g., the stimulus from recognition or non-recognition of the sound intended to be a name for the object, i.e., the rose), not only would the object be identifiable, but is identifiable by a unique property (as sweet as a rose) that has more meaning than its mere chemical classification. In this case, Shakespeare intended that a rose would always smell as sweet, with the wide range of emotional secondary meaning assuredly being intended (pleasing, agreeable, delightful, amiable, dear, beloved, Compact Abridged Dictionary, Random House, Special Second Edition, 1996, p. 1921). Therefore, the precise quality of the sweetness of the rose that would be smelled to so precisely identify a rose would have those precise primary and secondary stimulation effects, with both cognitive (recognition of rose, smelling sugar content) and emotional (being delighted, feeling amiable) were also the direct result of smelling a rose. Therefore, aromatherapeutic effect has been recognized, if not specifically defined with that terminology since the 16^{th} Century in English literature.

Non-industrial cultures are believed or presumed to share a greater affinity and day-to-day relationship with the use of natural materials, specifically even those useful only for the effects of their contact or stimulation of people. Such non-industrial cultures are often portrayed as having great horticultural expertise, knowing ‘traditional’ remedial effects of natural materials, and the differing uses of natural materials, including for ritualizing or exercising an emotional control over the feelings of persons stimulated by the emotional impact of words upon many listeners. The actual ability to control or effect emotions by appropriately selected stimuli therefore cannot be denied.

Studies have shown that ambient odors can reduce anxiety and change emotions (King, J. R., Perfumery: The Psychology and the Biology of Fragrance, Van Toller and Dodd (eds.), London: Chapman and Hall, Ltd., 1988, pp. 147-165). Schiffman describes a study in which patients were conditioned to associate a certain odor with a relaxed state. Patients were able to reduce the severity of their anxiety episodes by inhaling their designated fragrance (Schiffman, S., Fragrance: The Psychology and Biology of Perfume, Van Toller and Dodd (eds.), London: Elsevier Applied Science, 1992, pp. 57-58). In a study by Hirsch on the relationship of odors and perceptions of room size, the subjects perceived the size of a small booth to the larger after inhaling a scent similar to green apples. Hirsch speculated that the green apple scent reduced the anxiety of being enclosed in a small space and thereby increased the perceived room size (Hirsch, et al., Manuscript 1994: 2).

Some of these psychological effects (or at least physiological activities that are associated with physiological or psychomotor conditions) are even asserted to be measurable as shown in studies that attempt to quantify an average normal threshold for various odorant substances. For example, see Amoore and O'Neill, “Proposal for Unifying Scale to Express Olfactory Thresholds and Odor Levels: The “Decismel Scale”,” in Proceedings of the 1988 Air Pollution control Association Annual Meeting, Paper No. 78.5 (21 pp.), Air and Waste Management Association, Pittsburgh, Pa. (1988); Amoore and Haotala, “Odor as an Aid to Chemical Safety: Odor Thresholds Compared with Threshold Limit Values and Volatiles for 214 Industrial Chemicals in Air and Water Dilution,” J. Appl. Toxicology 3(6):272-290 (1983), which was related to aromatherapeutic responses that were measured by association with specific physiological values that can be measured. For example, U.S. Pat. Nos. 4,670,264; 4,670,463; and 4,671,050 (all these patents referred to collectively as Warren et al.) describe compositions and methods of causing the reduction of physiological and/or subjective reactivity to stress in humans being subjected to stress conditions. The Warren et al. patents describe a method for reducing physiological and/or subjective reactivity to stress in humans being subjected to stress conditions. The method of the Warren et al. patents consists of administering to such humans an effective amount of a physiological and/or subjective stress reactivity-reducing substance selected from the group consisting of: (i) Nutmeg Oil; (ii) Mace Extract; (iii) Neroli Oil; (iv) Valerian Oil; (v) Myristicin; (vi) Isoelemicin; and (vii) Elemicin. Administration is through inhalation or transdermally using one or more of the above ingredients alone or in a suitable composition such as ethanol and/or a perfume composition, cologne or perfume article (e.g., air freshener or deodorant stick). Also described is a method for detecting the reduction of physiological and/or subjective reactivity to stress in a human.

There are many different discloses of methods and compositions for the delivery of natural materials for the purposes of aromatherapy. These methods usually encompass the ingestion (eating or drinking) of the natural material (fruit, grain, leaf, oil, juice, or substance or distillate of the material), burning of the material to provide the actives to the ambient environment, applying a composition containing at least the active ingredients from the natural material directly to the subject (e.g., perfume, lotion, cream, soap, abrasive composition, etc.), or otherwise placing the composition in a location or system (e.g., a vaporization system) that would provide an active amount of the natural material to the ambience of the location. This may be as direct a targeting of an environment as a car air freshener.

Essential Oils comprise a class of material that is well recognized by the relevant art (cf, “The Encyclopedia of Essential Oil” Julia Lawless, ELEMENT, Boston, Mass., 1992). These materials, just as are most herbs, essences, powders, spices, fragrances and the like, are derived from living organic systems, that is, plants and animals. They may be found in large quantities in organic sources, as with the large volume of orange oil available in orange peel, or they may be found in minute quantities in specific portions of a plant, as with rose oil (where one ton of rose petals is needed to produce 300 grains of rose oil, or that there is less than one part of rose oil to every 48,000 parts by weight of rose petal, cf, Lawless, supra, pp. 11-15). Almost all oils listed are recognized as having various properties. Some are also identified as antiseptic or bactericidal (e.g., Citronella, Cymbopogon nardus (Lawless, p. 83); Eucalyptus Blue Gum, Eucalyptus globules var. globules, (Lawless, p. 93); Lavender, True, Lavandula augustifolia (Lawless, pp. 117-18); etc.) as well as having other described effects or uses.

For all of this knowledge of the properties of essential oils and the many benefits that they may contribute, there are still new ways in which these materials may be used that have not been appreciated by this residue of knowledge.

Among the many known uses and formats for using aromathetrapeutic materials are the following disclosures. U.S. Pat. No. 6,045,813 (Ferguson, et al.) describes a flowable personal care or cleaning composition, comprising a carrier and friable beads disbursed in the carrier, the beads containing an active ingredient and enclosing the active ingredient in a wall of bead material, the active ingredient amounting to approximately 0.5-5.0% by weight of the bead including its wall material and the active ingredient, wherein the active ingredient is selected from the group consisting of fragrance, bactericidal liquid, a pharmaceutical, a skin moisturizer and a cleanser, the carrier having a different color from the colorant in the wall material of the beads and the beads amounting to between 0.5 and 10% by weight of the composition. The reference describes that disclosed dimensions are representative for the beads used in the lotions of the present invention, both the essential oil containing lotion referred to as an “Aromatherapy” lotion, and an antibacterial lotion containing Triclosan or other antibacterial agent.

U.S. Pat. No. 5,785,972 (Tyler) describes a composition of matter comprising a therapeutically active compound with antiseptic and osmotic characteristics for treatment or therapy for burns and open wounds experienced by animals and man and in particular to the treatment of thermal burns on humans by use of spray, mist, dropper or saturated bandage application of the solution disclosed. The compound in solution form composed of colloidal silver, helichrysum angustifolium or helichrysum italicum oil and raw honey emulsified with water soluble lecithin by agitation. Helichrysum angustifolium and italicum (also referred to as immortelle and everlasting) are natural plant oils and are recognized as essential oils with properties promoting the healing process, in human and animal, of traumatic wounds including burns. Helichrysum is the distilled oil produced from flowering heads of Helichrysum Angustifolium D.C. or Italicum and is generally known for the ability to enhance human or animal wound healing and is generally accepted to have antispasmodic, analgesic, antiseptic and anti-inflammatory characteristics in relation to treatment of human or animal trauma. It is also reported to abate bleeding from wounds and in the reduction of scar tissue (The Complete Book of Essential Oils & Aroma-Therapy, pages 9, 400, Valerie Ann Worwood, published by New World Library, ISBN 0-931432-82-0; Aromatherapy Workbook, pages 16, 36, 58 and 60, published by Healing Arts Press, ISBN 0-89281-346-6; Portraits in Oils, Philippe Mailhebiau, page 65, published by Saffron Walden, ISBN 0-85207-237-6). Helichrysum is available commercially. Raw honey is known to have antiseptic qualities (A Disaster Survival Guide, Cass Ingram, D. O., page 26, published by Literary Visions Publishing, Inc., ISBN 0-911119-44-2; Scientific American, December 1996, page 102). These references are provided herewith in an Information Disclosure Statement in accordance with 37 CFR 1.97.

U.S. Pat. No. 5,382,567 describes aromatic compositions which comprise perfumes included in cyclodextrins whose inclusion ability depends on a pH of a solution containing the perfumes and cyclodextrins and pH-adjusting substances whereby a release rate of the perfume can be controlled, are described. Aromatic compositions are also disclosed, which comprise perfumes coated or covered with materials whose solubility depends on the pH of a solution containing the coated perfumes, and a pH-adjusting substance to change the pH as desired. Methods for controlling the release rate of the perfumes are also described.

U.S. Pat. No. 5,498,637 (Timmermeyer, Sr., et al.) relates to a method for moisturizing the skin with a natural product consisting of fresh lemon juice, extra virgin olive oil, and water. The substance of the invention is the extra virgin olive oil. The extra virgin olive oil, lemon juice and water are all natural substances that have been proven for centuries to be beneficial to humanity. The medical field, health occupations, beauty and cosmetics industry, Aromatherapy, Herbalism and gourmet cooks, chefs, and lay people can and have continued to recognize these ingredients to enhance their lives. The lemon cleanses acid and dirt off of the skin while the extra virgin olive oil moisturizes the skin and brings it back to a smoother texture with a clean natural glow. The water cools and blends the combination of lemon and oil so that the mixture can be easily absorbed into the skin. Body skin, feet and elbows and the entire body skin will become smoother and softer.

U.S. Pat. No. 5,725,833 (Crafton) describes a water buffered essential oil non-open flame incensing system including a level fire plate with a metallic fire ring centrally bonded to the upper surface thereof. A stoneware urn is provided having a body portion with a pronounced belly, a bell lip, a throat defined between the body portion and the bell lip, a curved interior surface with a non-flat bottom portion, and an exterior surface. At least the curved interior surface has high fire glazed finish. Water and essential oils to be diffused are filled in the urn and the urn is placed on top of a burning charcoal briquette on the fire ring. Boiling of the water carries the oil in the form of droplets into the atmosphere. The urn is sized to have a small amount of water left at the time the charcoal burns out. A smokeless system may be beneficial for people with smoke allergies.

U.S. Pat. No. 5,607,409 describes a delivery system comprising a steaming device for skin treatment includes a base, having an upper and lower surface, the base including a reservoir for water and a non-corrodible conductance element which is defined by two side-by-side vertically positioned electrodes which extend a part of the height of the reservoir. The electrodes diverge from each other as they approach the bottom of the bottom of the base to reduce current flow between them, this compensating for increased conductivity due to mineralization of the water near the bottom of the reservoir that occurs with vaporization and which mineralization would otherwise increase voltage between electrodes to a hazardous level. The device further includes a top portion including an upper and a lower area, and a container for therapeutic substances to be mingled with steam produced by the effect of the conductance element on the water. The device also includes an arm having a proximal and distal end, and hollow interior, in which the arm communicates with the top portion at the arm's proximal end and in which the arm includes an expulsory aperture enabling steam to escape at the distal end of the arm.

U.S. Pat. No. 5,620,695 describes a method and composition for the treatment of minor skin irritations based upon the use of a carrier oil with the essential oils of eucalyptus, lavender and ti tree added to the oil with a most unique composition capable of reducing the irritation, promoting healing, resisting insects, and take advantage of the science of aromatherapy. This lotion provides antiseptic and antibiotic properties to inhibit infection, has soothing and calming qualities to decrease the irritation and prevent animals from scratching further, repels insects which decreases infection and itching, cools and irritation and speeds healing. Its advantages are: it easy to apply and will benefit almost any skin irritation, it has no known side effects, it is all natural, its odor repels insects, it is not changed or rendered ineffective in any weather conditions, it can be applied by hand with non-sterile technique and the type, qualify and composition of all ingredients are considered safe by human standards, and indeed, are often used in massage creams by professionals. The reference describes that essential oils are usually added to a carrier oil, like canola, that serves as a stabilizing and diluting base the encourages absorption in addition to coating the skin. Lavendula angustifola (lavender) is considered the most useful and most versatile therapeutically of all the essential oils and is effective on almost all skin conditions. It is considered antiseptic, antibiotic, analgesic and anti-inflammatory. It also has been used historically as an insect repellent. Research found that it is cytophylactic—i.e., it encourages skin cell regeneration. It is considered safe enough to use on human infants. It has the additional emotional effects of calming emotions and nervous tension. Eucalyptus globulus cools the skin. Its odor repels insects. It is anti-inflammatory and highly effective on fungal infections. It was used in World War II as an antiseptic. Ti tree, also spelled tea tree but correctly names Melaleuca Alternifolia, has the unusual properties of being antifungal, antiviral and antibacterial.

U.S. Pat. No. 6,106,837 describes a method for preventing or reducing the symptoms and/or duration of a migraine or other form of headache through the administration of a hedonically pleasant odorant that is inhaled by a person who is prone to or suffering from a headache. A preferred odorant is one having the characteristics of a green apple scent. Preferably, the person is presented with the odorant at a suprathreshold concentration, and inhales the odorant for about 5-15 minutes while in a prone position in a quiet, darkened room. Preferably, the subject individual is presented with the odorant at a suprathreshold concentration (e.g., about 25-55 decismel units), and inhales the odorant for about 5-15 minutes, preferably about 10 minutes. It is further preferred that the individual inhales the odorant while in a prone position, preferably in a quiet room and one that has subdued lighting or, preferably, is completely darkened.

U.S. Pat. Nos. 4,670,264; 4,670,463; and 4,671,050 (all these patents referred to collectively as Warren et al.) describes compositions and methods of causing the reduction of physiological and/or subjective reactivity to stress in humans being subjected to stress conditions. The Warren et al. patents describe a method for reducing physiological and/or subjective reactivity to stress in humans being subjected to stress conditions. The method consists of administering to such humans an effective amount of a physiological and/or subjective stress reactivity-reducing substance selected from the group consisting of: (i) Nutmeg Oil; (ii) Mace Extract; (iii) Neroli Oil; (iv) Valerian Oil; (v) Myristicin; (vi) Isoelemicin; and (vii) Elemicin. Administration is through inhalation or transdermally using one or more of the above ingredients alone or in a suitable composition such as ethanol and/or a perfume composition, cologne or perfume article (e.g., air freshener or deodorant stick). Also described is a method for detecting the reduction of physiological and/or subjective reactivity to stress in a human. These Warren et al. Patents also describe that it is known in the art that a “normosmic” individual is one who can detect the odor of a substance without irritant sensations when the odorant is presented with the range of its average normal threshold. A “hyposmic” or “microsmic” individual has reduced capacity of the olfactory nerve being able to detect an odorant substance by its odor at a concentration, or decismel level, above that of a normosmic individual yet below its irritant concentration level. An “anosmic” individual is one who has essentially no olfactory nerve capacity, being unable to detect the odor of the odorant substance, but has trigeminel nerve function, being able to detect an odorant substance by means of irritant, tingling sensations when it is present at an irritant concentration. A patient who is able to detect pyridine vapor by means of irritant, tingling sensations caused by stimulation of the trigeminel nerve, but who cannot distinguish a pyridine odor at a lower concentration without such sensation, is considered to be anosmic having no olfactory nerve sensitivity.

Warren et al. also relate these stimulations to relief of stress. The term “stress” referring to an event or experience in the life of an individual that has specific physiologic and/or subjective consequences that disturb the equilibrium of the individual (Glock, C. Y. & Leonard, H. L., Journal of Chronic Diseases, 1956, 5, 179). Sources of stress may be an individual's occupation, for example, controlling air traffic at a busy airport, or it may be a life event change such as a change of job, a death in the family or a divorce, or it may be the small irritations and strains of everyday life—the daily hassles. The term “reactivity” is also defined as to the change generated by stress in the individual's physiologic and/or subjective condition. Within the context of this invention, “reactivity” may be ascertained objectively by measuring change in systolic blood pressure relative to equilibrium blood pressure resulting from the application of stress. The term “physiologic change” has been employed herein to identify this form of reactivity.

Unlike a drug that is ingested orally or injected subcutaneously, the substances used for the practice of the Warren et al. invention are inhaled and/or absorbed by means of transdermal penetration. Hence, the term “amount administered” meant “amount of stress reactivity-reducing composition calculated to have been breathed in, retained and absorbed into the bloodstream or transdermally absorbed into the bloodstream.” In measuring specific physiological effects, a statistically significant increase in systolic blood pressure (p<0.1), was generally 3 mm/Hg or more due to the stress. The subjective reactivity to stress was defined as a statistically significant change (p<0.05) in the individual's self-report report of one or more of the following emotions: Decrease in degree of relaxation; Decrease in happiness; Decrease in calmness; Increase in fear; Increase in tenseness; Increase in embarrassment; Increase in anger; and/or Increase in anxiety.

U.S. Pat. No. 5,960,506 (Reuven) describes a new pumice scrubbing pad for removing rough and dead skin from feet. The inventive device includes a plurality of filaments which are aggregated to form a pad member having spaces therein. Provided on the outer surface of the filaments is a scrubbing coating. The scrubbing coating includes a plurality scrubbing particles, a soap, a skin moisturizing agent, a fragrance, and a binder which couples the scrubbing coating to the outer surfaces of the filaments.

There seems to be a clear demarcation in the art that has been reviewed with respect to the use of natural oils for aromatherapy, and that division is that the essential oils generally, and the antibacterially active oils in particular, have been used by ingestion (in part or in whole, as with a beverage), surface contact, or environmental release solely for the release of air-born components.

Additionally, the use of synthetic antibiotics in household systems has created issues of an epidemiological nature. Bacteria are known to develop into strains of antibiotic resistant forms after repeated and minimally effective exposure to antibiotics. Misuse or insufficient use of antibiotics prescribed for therapy is one way in which ideal exposure conditions exist that can lead to the development of antibiotic-resistant strains. The use of synthetic antibiotics or elatives of orally or topically applied antibiotics in such a large variety of household products (soaps, shampoos, surface cleaners, dish cleaners, air sprays, and the like) raises the potential for a genuine concern that such a broad use of weak levels of antibiotics over such a wide range of bacteria and conditions is clearly a doubtful way to avoid increasing the possibility of generating strains that are either resistant to specific bacteria or which have been trained to more easily develop resistance to other bacteria.

It is therefore desirable to provide products that are capable of providing both household functions and antibacterial effects with a reduced likelihood of stimulating the development of immunity in specific bacteria to prescription, synthetic antibiotics.

### BRIEF DESCRIPTION OF THE INVENTION

A liquid household product is provided that has a functional application on a surface to which it is applied and after appropriate use of that household product on the surface, residual essential oils in the household product remain on the surface and provide aromatherapeutic content to an ambient environment around that surface. To that end, a household composition (for non-limiting examples, selected from the group consisting of surface cleaners (countertop cleaners, ceramic cleaners, glass cleaners, wood cleaners, window cleaners, carpet cleaners, wax cleaners and the like), antistatic compositions (sprays, liquids, wipes, etc.), dish soaps and dish cleaners, ironing liquids, wood finishes, anti-stain compositions, and the like. The product comprises the household product carrier base, with the essential oil as both an active agent (e.g., antibacterial agent) and as an aromatherapeutic ingredient (that persists for an effective amount of time after application of the product to an inanimate surface).

### DETAILED DESCRIPTION OF THE INVENTION

In the overpopulated and complicated, present-day community, attention has been recently drawn to adverse influences of stress on human body. Under these circumstances, various studies have been made, including investigations on amenities with which life space is made comfortable, developments of related substances, and countermeasures against offensive odor and public nuisance. Especially, aroma greatly influences human physiology and psychology. Aromatherapy which makes use of the effects of herbs, populi and phytoncides has been extensively made in various fields including psychologic medicines such as on stress as well as aromatic science and chemistry.

Specific examples of the perfumes include animal perfumes such as musk oil, civet, castreum, ambergris, plant oils such as sandalwood oil, citronella oil, ylang ylang oil, neroli oil, bergamot oil, lemon oil, lavender oil, sage oil, rosemary oil, peppermint oil, eucalyptus oil, verbena oil, citronella oil, cajuout oil, salvia oil, clove oil, petuli (alternatively spelled as ‘petchouli’) oil, citrus/mint oil combinations, chamomille oil, sandalwood oil, costus oil, labdanum oil, broom extract, carrot seed extract, jasmine extract, mimosa extract, narcissus extract, olibanum extract, rose extract and the like. Although some natural, non-essential oil ingredients and chemical substances such as acetophenone, dimethylindane derivatives, naphthalene derivatives, allyl caprate, alpha-amylcinnamic aldehyde, anethole, anisaldehyde, benzyl acetate, benzyl alcohol, benzyl propionate, bomeol, cinnamyl acetate, cinnamyl alcohol, cumin aldehyde, cyclamen aldehyde, decanol, ethyl butyrate, ethyl caprate, ethyl cinnamate, eugenol, geraniol, hexenol, alpha-hexylcinnamic aldehyde, hydroxycitrolnellal, indole, iso-amyl acetate, iso-amyl iso-valerate, iso-eugenol, linalol, linalyl acetate, p-methylacetophenone, methyl anthranilate, methyl dihydrojasmonate, methyl eugenol, methyl-beta-naphthol ketone, methylphenylcarbinyl acetate, musk ketol, musk xylol, 2,6-nanodinol, .gamma.-nanolactone, phenylacetoaldehydodimethyl acetate, beta-phenylethyl alcohol, 3,3,5-trimethylcyclohexanol, gamma-undecalactone, undecenal are known as perfume carrier or fragrances, they are not intended to be within the scope of the aromatherapeutic materials in the practice of the present invention.

The amount of the aromatherapeutic to be used in the residual material is generally the range of from 0.01 to 50 or 100 wt %, preferably from 0.1 to 30 wt %, based on the total weight of the aromatic ingredients in the composition after application to a surface for a primary household function.

Previous technologies have offered numerous means of introducing chemical aromas into a room using methods that produce a wide range of intensity and duration. These means have typically employed imprecise techniques, such as opening and closing a hooded container as in McCarthy U.S. Pat. No. 4,603,030, spraying an approximate quantity of a chemical with an easily contaminated vaporizer as in Laube U.S. Pat. No. 2,905,049, or heating a liquid permeated material or compound as in Lee U.S. Pat. No. 5,398,070.

Reactivity to stress varies with the individual. Some individuals thrive on stress whereas in other individuals, the same stress drives them towards sickness (Executive Fitness Newsletter, Rodale Press Inc., Vol. 15, No. 17 [1984]). “Reactivity” hereinafter refers to a negative reaction to stress.

“Reactivity” to stress should not be confused with the abnormally high base-line blood pressure or anxiety levels which may require drug treatment. For example, it should not be confused with hypertension which is defined as blood pressure that remains consistently above 140 mm/Hg systolic and 90 mm diastolic pressure with repeated blood pressure determinations over the course of several weeks (Report of the Joint National Committee on Detection Evaluation and Treatment of High Blood Pressure; Journal of the American Medical Association, 1977, 277, 255-261).

It is appreciated that reactivity and hypertension or state of anxiety may be related. Abnormal anxiety or depression has its major primary causative components the fundamental conditions of helplessness, uncertainty, anticipation, undirected arousal and the like (Zucker, M. & Spielberger, C. D., “Emotions and Anxiety”, Lawrence Erlbaum Associates, Publishers; Hillsdale, N.J. [1976]). See, also, The Diagnostic and Statistical Manual of the American Psychiatric Association, DSM3, 1980.

Our invention for stress reactivity reduction utilizes plant-derived substances common to the fields of perfumery and aromatherapy. The dose levels, however, differ from those normally employed in either perfumery or aromatherapy and the mode of application differs from those normally employed for aromatherapy and are significantly less. In addition, concern over (minor) blood pressure surges and possibilities for damping such surges appear to be absent in the folk medicine literature.

The term “Aromatherapy” is intended herein to mean the use of plant-derived substances; volatile substances derived from plants for the treatment of health problems. Generally, the volatile fraction—the essential oil fraction—of the plant-derived substance is used. The use of the volatile fractions of plants for treatment of various ailments is reviewed in the following three monographs:
(1) J. Valnet, “The Practice of Aromatherapy”, Destiny Books (Division of Inner Traditions International, Ltd.), New York, N.Y., 1982;
(2) R. Tisserand, “The Art of Aromatherapy”, Destiny Books (Division of Inner Traditions International, Ltd.), New York, N.Y., 1983; and
(3) A. Leung, “Encyclopedia of Common Natural Ingredients”, J. Wiley & Sons Publishing Co., New York, N.Y., 1980.

A detailed analysis of the aromatherapy folk medicine literature suggested that a number of essential oils commonly used in perfumery might have a multiplicity of medical effects. Some of these oils are employed in the practice of this invention. Neroli oil is the essential oil obtained from orange blossoms. Neroli oil has a folk medicine history as being an anti-depressant, aphrodisiac, antiseptic, antispasmodic and of having digestive and sedative activity. The anecdotal literature suggests that neroli oil is an effective sedative and anti-depressant and that it may be used for insomnia, hysteria, states of anxiety and depression (R. Tisserand, “The Art of Aromatherapy”, cited, supra). Tisserand further states: “Neroli is one of the most effective sedative-antidepressant oils: it may be used for insomnia, hysteria, states of anxiety and depression. It calms and slows down the mind. It also has a notable action on the heart, diminishing the amplitude of heart muscle contraction, hence its use in palpitations or other types of cardiac spasm. Derived from this is its use in panicky, hysterial, fearful types of people—those who upset themselves unnecessarily, and become over wrought over nothing. One can also see that neroli is a valuable remedy for shock, or for disorders caused by sudden shock, or fear, causing a strain on the heart. It is valuable in chronic diarrhoea, when this is related to long-standing stress or fear. Its action is slow but sure.”

Oil of neroli also has a pronounced action on the skin. Like lavender and geranium, it can be used with benefit on any type of skin. It is totally non-irritant and may be used where there is irritation or redness. It is said to be useful for dry skin and broken veins. It is one of the oils which acts on a cellular level stimulating the elimination of old cells and the growth of new ones. Neroli makes a luxurious, relaxing, and deodorant bath oil.

Orange-flower water is soothing, digestive, carminative. It makes a very useful, mild remedy for infants' colic, and its sedative action helps to send people, especially small children, to sleep.

Ylang Ylang (Cananga odorata var. genuina) of the Annonaceae is derived from the flowers of a tropical tree native to tropical Asia. In Indonesia, the flowers are spread on wedding beds. In the Molucca Islands, an ointment made from Ylang Ylang and cucuma flowers is used in a coconut oil base for cosmetic care, hair care, skin diseases, fever reduction (including from malaria), and fight infections.

Valerian oil is the essential oil obtained from the root of Valeriana officinalis. The folk medicine literature lists the valerian root (fresh or dried) as being useful as an antispasmodic, carminative, stomachic and sedative. It has been used to treat migraine, insomnia, hysteria, fatigue and stomach cramps that cause vomiting (A. Y. Leung, “Encyclopedia of Common Natural Ingredients”, John Wiley & Sons, New York, N.Y., 1980, pages 317-320).

Regarding valerian oil's use in Russia, Hutchens, et al, “Indian Herbalogy of North America”, (published by Merco of Windsor, Ontario, Canada), 5th edition, 1974, states: “Russian Experience: Valeriana is known to Folk Medicine as having a general calming and sedative effect on the central nervous system, to induce sleep and rest, spasms of the stomach, intestines and blood vessels, nervous heart conditions. Further acknowledgement as appetizer, headache relief, hysteria, epilepsy, tape worm, diarrhea, lose stomach, fever. Externally: Vapour baths given to children will quieten and encourage restful sleep (Bello-Russ. Academy of Science, Minsk, 1965).”

In “Sedative Principles of Valeriana Roots”, Hikino, et al, Shoyakugaku Zasshi, 34, (1), 1980, pages 19-24, it is indicated that compounds possessing sedative activity, isolated from valerian roots, have failed to fully account for the sedative activity exhibited by the roots per se. It is further stated therein that, recently, iridoids named valepotriates were isolated as analgesic and sedative principles from Indian valerian roots. In this paper, a correlation between the contents of the valepotriates and the pharmacological activity of various valerian roots was examined. Napalese and Chinese valerian roots containing an appreciable quantity of valepotriates showed no sedative activity, while Japanese valerian root containing less valepotriates inhibited stress-induced ulcer formation and prolonged hexobarbital-induced sleep in mice. An extract of “Hokkai-kisso”, i.e., roots of a Japanese valerian, was fractionated and the effect of each of the fractions on the enhancement of hexobarbital anesthesis was tested. Kessyl glycol diacetate, Kessyl glycol 8-acetate and Kessyl glycol 2-acetate were obtained as active principles therefrom. The enhancement of hexobarbital anesthesis by Kessyl glycol diacetate was assumed to be due to its inhibitory effect on the central nervous system. Kessyl glycol diacetate exhibited no inhibitory action on the stress-induced ulcer production.

The chemical constituents, pharmacology and known uses of valerian are reviewed in: “Herbal Remedies Used in Sedative and Antirheumatic Preparations: Part I”, Phillipson, et al, The Pharmaceutical Journal, Jul. 21, 1984, pages 80-82.

Another potentially interesting plant substance is nutmeg which was important in medicine as well as well as in cooking. It was used as a therapeutic by Arab physicians as early as the 7th Century A.D. for treatment for disorders of the digestive system, kidney disease, pain and lymphatic ailments. Nutmeg is a significant item in the Hindu Pharmacopeia wherein it has been prescribed for fever, consumption, asthma and heart disease. Nutmeg is employed by folk practitioners in India as an analgesic and sedative. In large doses (two teaspoons or more of ground nutmeg), nutmeg exhibits mild hallucinogenic activity, the description by Payne (R. B. Payne, New England Journal of Medicine, 269, pages 36-38 [1963]) being illustrative of this activity. Two college students, 19 and 20 years old, each consumed two tablespoons (14 grams or the equivalent of two whole seeds) of powdered nutmeg in milk. About 5 hours later, each had the onset of a leaden feeling in the extremities and a nonchalant detached mental state described as “unreal” or “dreamlike.” Rapid heart rates and palpitation were observed and both complained of dry mouth and thirst. See, also, Wiel (A. T. Weil, Ethnopharmacol. Search Psychoact. Drugs [Proc. Symp.], 1967, [Pub. 1979], 188-201).

The fraction of nutmeg responsible for the mild hallucinogenic activity is suggested by the literature to be the aromatic fraction of the oil containing safrole, methyleugenol, eugenol, methylisoeugenol, myristicin, elemicin, isoelemicin and methoxyeugenol as the major components. Of these, myristicin, elemicin and isoelemicin have been reported to be the active molecules (A. T. Shulgin, et al, Ethnopharmacol. Search Psychoact. Drugs [Proc. Symp.], 1967, [Pub. 1979], 202-214). The myristicin-elemicin fraction of oil of nutmeg produces many of the activities of crude ground nutmeg but lacks adequate potency to explain the nutmeg intoxication syndrome on a quantitative basis. Nutmeg and synthetically made myristicin show a mild degree of monoamine oxidase inhibiting activity. The monoamine oxidase activity is found in the volatile component of nutmeg (E. B. Truitt, Jr., Ethnopharmacol. Search Psychoact. Drugs [Proc. Symp.], 1967, [Pub. 1979], 215-222).

Nutmeg oil, known as myristica fragrans, or myristicaceae, is the essential oil from the kernel of the fruit of the nutmeg tree. The stone of the fruit is enclosed within a husk which, when dried, is known as mace. “Mace Extract” is an aromatic essence extracted from mace. “Nutmeg Butter” is a fixed oil obtained by hot-pressing the nutmeg kernels, and contains myristine, butyrin, olein, palmitine and stearine. The essence contains 80% pinene and camphene, 8% dipentene, 6% terpenic alcohols, (linalool, borneol, terpineol and geraniol), 4% myristicin and various substances such as eugenol and safrol. Valnet, “The Practice of Aromatherapy”, (supra) states that, for external use:

(a) “nutmeg butter” is used in liniments for the treatment of rheumatic pains and toothaches; and

(b) “nutmeg butter” is used in the form of “nerve balm” for treatment of rheumatic pains, the form being a mixture of the essences of rosemary and clove together with nutmeg butter.

A form of nutmeg oil, Myristica castaneifolia (Myristacaceae) Fiji, is described as possessing biological activity, specifically in the anti-tumor field, in U.S. Letters Pat. No. 4,352,797 issued on Oct. 5, 1982, the specification for which is incorporated by reference herein. At page 4 of the January/February 1984 (Vol. 6, No. 1) edition of FOCUS (World Wildlife Fund-U.S.), nutmeg is indicated as being an analgesic and a hallucinogen. In the paper “Nutmeg as a Narcotic” by Kalbhen in Angew. Chem. 83, 379 (1971), Kalbhen discloses that the hallucinogenic ingredients of nutmeg include, inter alia:

Furthermore, Myristica fragrans is disclosed at Chem. Abstracts, Vol. 101, No. 2831g (abstract of Japan Kokai Tokkyo Koho 59/55,827) as being useful in the field of drug stabilization in conjunction with the utilization of transdermal pharmaceuticals. The said abstract and the said Japanese Kokai Tokkyo Koho are incorporated herein by reference.

Furthermore, isoelemicin is a known flavor ingredient as set forth in U.S. Letters Pat. No. 3,686,004 issued on Aug. 22, 1972, the specification for which is incorporated by reference herein. By the same token, myristicin is disclosed as a component of the aroma of blueberries in J. Sci. Food Agric., 1983, 34(9), 992-6 (abstracted at Chem. Abstracts, Vol. 99:174466r). (Hirvi, et al).

Furthermore, regarding myristicin, Arctander, “Perfume & Flavor Chemicals (Aroma Chemicals)”, published by the author in 1969, states at monograph 2291, that myristicin is: “Pleasant and warm-balsamic, slightly woody odor of good tenacity. The undiluted material shows some ‘pepperiness’. This material, although commonly found in natural oils, has found only limited use in perfumery . . . ”

The essential oils described above are also common perfumery ingredients as described in Arctander, “Perfume and Flavors Materials of Natural Origin,” published by the author in 1960. (Mace extract at columns 391-393; neroli oil at columns 435-437; nutmeg oil at columns 442-445; and valerian oil at columns 637-638).

The compositions useful in the practice of the combined household functions and aromatherapy comprise the oils in combination with carrying agents, including both liquid carriers and blends/solutions of liquid and solid-forming carriers (e.g., polymer binders, waxes, high viscosity agents, volatility reducing agents such as low odor solvents that are less volatile than the essential oils and will retard or control their volatilization. Other additives such as stabilizers (e.g., ascorbic acid for oxidation stability, UV absorbers for UV stability, free radical scavengers for storage stability, etc.; including but not limited to antioxidants, methylparaben, ethylparaben, hydroquinones, betaines, chelating agents particularly for metal ion scavenging, and the like), colorants, aroma modifiers, aroma masking agents, thickening agents (both organic, polymeric, inorganic, natural, and synthetic thickeners such as glycerin, acrylic polymers, corn starch, silica, kaolin clay, bentonite, salt, higher molecular weight oils, oil soaps), eutectic agents to retard volatilization, surfactants (e.g., to assist in liquid film spreading and flow properties; including but not limited to anionic surfactants, cationic surfactants, non-ionic surfactants, Zwitterionic surfactants, and mixtures thereof), sudsing and degreasing agents, solubilizing agents, gels, cleansing or cleaning agents (e.g., ammonia-D for window solutions or ethanol for hygienic surfaces), etc. Because of the scope of properties that are available from the natural ingredients, the compositions of the invention, as later described in greater detail, can be formulated in a much more environmentally friendly format than many existing products intended for the same household functional use.

The proportions of materials that are used in the products vary depending upon the specific type of product that is being considered. For example, window cleaning compositions should have little substantive (solid—non-volatile film-forming) content, although the solution should not be so volatile as to leave the surface before it has been cleaned properly or to concentrate removed matter in the solution before the cleaning liquid is removed. On the other hand, without causing a significant waxy buildup, wood finishes should both finish and coat the wood in a more substantive manner. Ironing liquids, depending on whether a starch component is present, need little body or should leave little residue and should have no coloration. For environmental advantages and long-range health concerns, the compositions are preferably free of antibacterial agents, antibiotics, and the like except for those comprising the essential oils of the invention or those with other primary functions (e.g., an alcohol co-solvent or carrier, a fragrance modifier, stabilizer or the like). The compositions may also be free of phosphates, which are of environmental concern. The essential oil when applied to a surface for a household function, often (along with some other ingredients) will be absorbed by or adsorbed to the surface so that the diffusion of the essential oil into an ambient environment will occur over time.

By ambient environment, it is meant an environment where there is some significant confinement of the air in that region, such as a residence, business office, enclosed vehicle, kitchen, rest room, lavatory, theatre, museum, or the like. It would not apply to an outdoor stadium, park area, or the like. The ambient environment should not have all air in the volume replaced in less then fifteen minutes for the release of the aromatherapy benefits of the essential oils.

The process of the present invention would be practiced by applying a liquid composition to a surface within the ambient environment for a household purpose (e.g., surface cleaning, surface shining, degreasing, cleansing, foreign matter removal, moisturizing, surface moisture application, e.g., ironing liquids, dish soap, and the like. It is also possible for solid compositions such as bar soaps and liquid compositions for application to persons (such as hand soaps and face soaps) to be formulated according to the beneficial practices of the invention. The ingredients may therefore be selected from within a broad range of proportions and materials, based in great part upon the specific uses intended. With that broad limitation contemplated, a general range of ingredients would include 5-99% by weight of liquid carrier (e.g., water, alcohol, salt water, ammonia water, light organics [less preferred] and the like), 0.1 to 20% by weight of at least one essential oil, 0-5% colorants, 0-10% thickening agents, 0 to 5% stabilizing agents, 0-50% by weight volatilization suppressing components for the essential oils (e.g., forming a eutectic with the essential oil or causing a lower vapor pressure for the essential oil), and the like. The materials may be selected on the basis that all materials are natural (produced by plants and/or animals, without being derivatized or with minimal derivatization (e.g., forming an ester or salt of the original natural ingredient) and therefore more biodegradable than synthetics that have been designed to be more durable. That process can be summarized as a method for providing aromatherapy to persons within an ambient environment comprising applying a liquid composition to an inanimate surface to effect a household function, the liquid composition comprising an aromatherapeutic concentration of an aromatherapeutic essential oil, completing the household function, allowing the aromatherapeutic essential oil to remain within the ambient environment to effect aromatherapy on persons or animals within the ambient environment. The process can be initiated by a determination that a specific emotionally therapeutic intervention or stimulation is desired for individuals that will be present in an ambient environment. Upon determination of a prescribed need for specific emotionally therapeutic enhancement or therapy, the specific essential oil that has been determined to provide the specific therapy that is desired for the particular individuals that will occupy the ambient environment will be provided in the aromatherapy treatment.

There is another benefit from a single manufacturer designing a line of aromatherapeutic household compositions. The same functional ability may be designed into each household composition (that is all window cleaners will have the same cleaning performance, each iron spray will have the same wetting and starching ability, each wood finishing composition will provide the same durable shine, etc.), but the entire line may provide the identical aromatherapy environment to the ambient environment. That is, the home may use Ylang Ylang window cleaner, Ylang Ylang wood finish, Ylang Ylang iron spray, etc. so that there are no conflicting aromatherapy effects. In this manner, a specific aromatherapeutic effect can be targeted for the entire environment without an overwhelming number of competing effects and smells.

This effect would be accomplished by providing a line of products with the identical underlying aromatherapeutic properties (the same essential oil fragrance). For example, a line of products or a kit with a number of different products with the identical aromatherapeutic properties would be provided. The line or kits could include such various products as wood finishes, floor cleaners, ceramic cleaners (for sinks, toilets, baths, etc.), ironing liquid, bath soap, hand soap (solid and/or liquid), glass cleaner, metal surface cleaner, fabric softener, and the like. Such products would have the essential oil formulations developed for each component of the product line or kit. Examples of these types of products are provided below.

### EXAMPLES

| | | |
|---|---|---|
| Component | Actual | Range |

Formulation for a Lavender Liquid hand soap (Percent by weight)

| | | |
|---|---|---|
| Deionized water | 65% | 30-85% |
| Glycerin | 2% | 0.0-6% |
| Long chain aliphatic alcohol | 28% | 10-40% |
| Non-ionic surfactant | <1% | <5% |
| Anionic surfactant | <1% | <5% |
| Cationic surfactant | <1% | <5% |
| Tetrasodium EDTA | <1% | <5% |
| Cocoamidopropyl Betaine | 1% | 0.0-7% |
| Tween 20 | 1% | 0.1-5% |
| Olive oil | 1% | 0-5% |
| Sodium chloride | q.s. 3000-6000 cps | |
| Citric acid | q.s. to pH 6.5 +/− 0.25 | |
| Fragrance of Essential oil* | 1% | 0.2-20% |

Essential Oil Dishwashing Liquid

| | | |
|---|---|---|
| Deionized water | 25% | 15-55% |
| Long chain aliphatic alcohol | 73% | 50-85% |
| Actiphyte of Soap Bark | 0.1% | 0-2% |
| Tetrasodium EDTA | <0.1% | <5% |
| Colorants | <0.1% | <5% |
| DMDM Hydantoin (McIntyre) | 0.60% | <5% |
| Citric acid | q.s. to pH 7.0 +/− 0.25 | |
| Fragrance of Essential oil | 1.5% | 0.2-10% |

Composition of Linen Water (Ironing Liquid or Spray)

| | | |
|---|---|---|
| Deionized water | 98.7% | 80-99.5% |
| Surfactant | <0.1% | <2% |
| Solubilizer | <1% | <5% |
| Starch | 0% | <8% |
| Fragrance of Essential oil* | 0.5% | 0.1-10% |

Composition of Furniture Cream

| | | |
|---|---|---|
| Deionized water | 90% | 70-97% |
| Carbomer ® antistatic agent | <0.2% | 0.05-4% |
| Tetrasodium EDTA | <0.5% | <5% |
| Actiphyte of Lemon Balm | <0.1% | <2% |
| Carnauba wax | <0.5% | 0.1-2% |
| Beeswax | 1.0% | 0.2-4% |
| Jojoba Oil | 2% | 0.2-5% |
| Sorbitan Oleate | 2% | 0.2-5% |
| Polysorbate 80 | 2% | 0.2-5% |
| Silicone Emulsion (GE 2135) | 2% | 0.2-5% |
| Vinegar | 0.1% | 0-5% |
| Fragrance of Essential oil* | <1% | 0.2-15% |

Composition of All-Purpose Cleaner

| | | |
|---|---|---|
| Deionized water | 85% | 65-95% |
| Actiphyte of Birch Bark | 0.1% | 0.01-2% |
| Blend of anionic and non-ionic | 5.8% | 0.5-12% |
| surfactants | | |
| Sodium Citrate | 1.8% | 0.2-7% |
| Detergent | 1% | 0.2-5% |
| Colorant | <0.1% | |
| Triton X-100 (surfactant) | 2.2% | 0.2-5% |
| Essential Oil Fragrance | 1.1% | 0.1-10% |

Composition of Window Cleaner

| | | |
|---|---|---|
| Deionized water | 90.5% | 75-98% |
| SD 40 Alcohol (Milsolv) | 5.25% | 1-12% |
| Detergent | <1% | 0.2-4% |
| Solubilizer | <1.0% | 0.05-5% |
| Dowanol surfactant | 2.75% | 0.5-6% |
| Essential Oil Fragrance | 0.15% | 0.03-5% |

The terminology “q.s.” means quantity sufficient to effect the property identified.
*Indicates that the essential oil is provided in a carrier and may comprise from 10-99% by weight of the fragrance additive.

1-25. (canceled)

26. A kit comprising different household products for effecting aromatherapy to an ambient environment, the kit containing at least three liquid household products, each household product comprising a liquid composition comprising at least 0.2% by total weight of an antibacterial essential oil, a solvent and a separate ingredient that effects a household function selected from the group consisting of surface cleaning, cleansing, dish washing, polishing, or finishing, the essential oil in each household product within the kit comprising at least one identical essential oil, wherein the solvent consists essentially of water, alcohol or a mixture of water and alcohol.

27. The kit of claim 26 wherein the at least one identical essential oil comprises Ylang Ylang.

28. The kit of claim 26 wherein the at least one identical essential oil comprises citrus mint.

29. The kit of claim 26 wherein the at least one identical essential oil comprises petuli oil.

30. A method for providing aromatherapy to persons or animals within an ambient environment comprising directly applying a composition consisting of a liquid composition to an inanimate surface to effect a household function selected from the group consisting of surface cleaning, surface shining, degreasing, cleansing, dish washing with soaps, and wood finishing, the liquid composition comprising an aromatherapeutic concentration of an aromatherapeutic essential oil of 0.1 to 20% by weight of the liquid composition, completing the household function, allowing the aromatherapeutic essential oil to remain within the ambient environment to effect aromatherapy on persons or animals within the ambient environment, the solvents in said liquid composition consisting of materials selected from the group consisting of water and alcohols, wherein the liquid composition is selected from the kit of claim 26.

31. A method for providing aromatherapy to persons or animals within an ambient environment comprising directly applying a composition consisting of a liquid composition to an inanimate surface to effect a household function selected from the group consisting of surface cleaning, surface shining, degreasing, cleansing, dish washing with soaps, and wood finishing, the liquid composition comprising an aromatherapeutic concentration of an aromatherapeutic essential oil of 0.1 to 20% by weight of the liquid composition, completing the household function, allowing the aromatherapeutic essential oil to remain within the ambient environment to effect aromatherapy on persons or animals within the ambient environment, the solvents in said liquid composition consisting of materials selected from the group consisting of water and alcohols, wherein the liquid composition is selected from the kit of claim 26, wherein two separate and different compositions each consisting of a liquid composition having the same aromatherapeutic essential oil are in the kit and are applied to different inanimate surfaces to effect a different household function.

32. A method for providing aromatherapy to persons or animals within an ambient environment comprising:
a) providing a holder supporting at least three containers having separate and different liquid compositions in each container;
b) removing one of the at least three separate containers and directly applying a liquid composition from the removed container,
wherein the liquid composition in the removed container comprises an aromatherapeutic concentration of an aromatherapeutic essential oil of 0.1 to 20% by weight of the liquid composition, the solvents in said liquid composition consisting of materials selected from the group consisting of water and alcohols,
c) performing a household function consisting of applying the liquid composition in the removed container to an inanimate surface to effect a household function selected from the group consisting of surface cleaning, surface shining, degreasing, cleansing, dish washing with soaps, and wood finishing,
d) completing the household function, allowing the aromatherapeutic essential oil to remain within the ambient environment, and
e) effecting aromatherapy on persons or animals within the ambient environment.

33. The method of claim 32 wherein at least two of the separate containers comprise different liquid compositions and at least two of the different liquid compositions are selected from the group consisting of surface cleaners, surface shiners, degreasers, cleansers, and dish washer liquids.

34. The method of claim 32 wherein the essential oil is selected from the group consisting of as sandalwood oil, citronella oil, ylang ylang oil, neroli oil, bergamot oil, lemon oil, lavender oil, sage oil, rosemary oil, peppermint oil, eucalyptus oil, verbena oil, citronella oil, cajuout oil, salvia oil, clove oil, citrus/mint oil combinations, chamomille oil, costus oil, labdanum oil, broom extract, carrot seed extract, jasmine extract, mimosa extract, narcissus extract, olibanum extract, and rose extract.
